(19) United States
(12) Patent Application Publication
(10) Pub. No.: US 2003/0181713 A1
(43) Pub. Date: Sep. 25, 2003

(54) Processes for the synthesis of chloroadenosine and methylthioadenosine

(76) Inventors: Jayaram Kasturi Srirangam, San Diego, CA (US); James Edward Saenz, Chula Vista, CA (US)

Correspondence Address: Agouron Pharmaceuticals Inc., 10777 Science Center Drive, San Diego, CA, 92121, US

(21) Appl. No.: 10/367,361

(22) Filed: Feb. 14, 2003

Related U.S. Application Data
(60) Provisional application No. 60/361,284, filed on Mar. 4, 2002.

(51) Int. Cl. C07H 19/16

(52) U.S. Cl. 536/27.21

(57) ABSTRACT

An in situ process for preparing chloroadenosine is described, wherein adenosine in a non-aqueous solvent is reacted with a thionyl chloride and a pyridine to form a reaction solution; the non-aqueous solvent is exchanged with a lower alcohol, and a base is added to the reaction solution; and the resulting chloroadenosine is filtered, washed and dried. Additionally, a two-step process for the synthesis of methylthioadenosine using the chloroadenosine prepared in situ is described.

### CROSS-REFERENCE TO RELATED APPLICATION

This application claims the benefit of U.S. Provisional Application Serial No. 60/361,284, filed Mar. 4, 2002, which is hereby incorporated by reference in its entirety.

### FIELD OF THE INVENTION

The present invention generally relates to processes for the synthesis of derivatives of adenosine. In particular, the present invention relates to processes for the synthesis of chloroadenosine and 5′-deoxy-5′-methylthioadenosine (referred to herein as “MTA”).

### BACKGROUND OF THE INVENTION

MTA, also known as vitamin L2, is the principal structural component of the biological methyl donor, adenosylmethionine, which is formed by enzymatic cleavage in a variety of reactions. MTA, a derivative of adenosine, promotes secretion of milk and is used in various fields of pharmacology. For example, MTA is an inhibitor of several S-adenosylmethionine (referred to herein as “SAM”) dependent methylations (Law et al., Mol. Cell Biol., 12:103-111, 1992). MTA has also been reported to be an inhibitor of spermine and spermidine synthesis (Yamanaka et al., Cancer Res., 47:1771-1774, 1987). Vermeulen et al. also discloses MTA as a methylation inhibitor used for treating non-viral microorganism infection (U.S. Pat. No. 5,872,104). MTA may also be used as a type of SAM metabolite that aids in the repair of connective tissue (U.S. Pat. No. 6,271,213 B1). Therapeutic uses of MTA as anti-inflammatories, antipyretics, platelet antiaggregants and sleep inducers are also known, as described in U.S. Pat. Nos. 4,454,122, 4,373,122, and 4,373,097. European Patent No. 0387757 discloses utilizing MTA in compositions favoring hair growth in subjects suffering from baldness, and European Patent No. 0526866 discloses utilizing MTA in the preparation of pharmaceutical compositions for the treatment of ischemia. Additionally, MTA can be used as an agent for the treatment of topical disorders, most notably, venous ulcers (Tritapepe et al., Acta Therapeutica, 15: 299, 1989).

There are several methods available for the synthesis of MTA. For example, MTA has been shown to be a product of spermidine biosynthesis in purified enzyme preparations from E. coli. However, MTA cannot be isolated in crude enzyme preparations because it is rapidly metabolized (Tabor and Tabor, Pharmacol. Rev., 16: 245,1964).

Several U.S. Patents also recite different synthetic methods for producing MTA. See, for example, U.S. Pat. Nos. 4,454,122; 4,373,097; and 4,948,783.

The MTA used in most biochemical studies has been obtained by acid hydrolysis of SAM (Arch. Biochem. Biophys., 75: 291, 1958; J. Biol. Chem. 233: 631, 1958). However, SAM is available only in limited amounts at considerable cost. A more economical process for the synthesis of MTA is therefore desired.

A two-step synthetic method for producing MTA is also known. Kikugawa et al. discloses a two-step synthesis method for producing MTA from chloroadenosine by reaction with alkyl mercaptants in the presence of aqueous sodium hydroxide (Kikugawa et al., Journal of Medicinal Chemistry, Vol. 15, No. 4, 387-390, 1992). However, the yield reported by Kikugawa is only 50-70% MTA.

Robins et al. discloses a synthetic method for producing MTA by conversion of adenosine via the intermediate 5′-chloro-5′-deoxyadenosine in a two-step reaction (Robins, Morris and Wnuk, Stanislaw, Tetrahedron Letters, 29; 45, 5729-5732, 1988; hereinafter “Robins I”). Robins I discloses a reaction scheme in which: (a) adenosine is reacted with thionyl chloride and pyridine in acetonitrile to form a cyclic intermediate which is then treated with ammonia, methanol and water to yield 91% chloroadenosine, and (b) MeSH, sodium hydride and dimethylformamide (“DMF”) is added to the chloroadenosine, resulting in the formation of MTA. Robins I, however, does not disclose the reaction conditions for carrying out the synthesis.

In a subsequent article, Robins et al. discloses the synthesis of MTA utilizing a three-step process for the conversion of adenosine to MTA. (Robins et al., Can. J. Chem. 69, 1468-1494, 1991; hereinafter “Robins II”). The three-step process described by Robins II included: (1) treatment of a stirred suspension of adenosine with thionyl chloride and pyridine in acetonitrile at 0° C., followed by warming to ambient temperature and isolation of a mixture of 5′-chloro-5′-deoxy-2′,3′-O-sulfinyladenosines intermediates; (2) treatment of the isolated mixture of intermediates with aqueous methanolic ammonia at ambient temperature to achieve deprotection and yield chloroadenosine (63%); and (3) treatment of chloroadenosine with thionyl chloride in DMF to yield only 54% MTA based on the initial starting materials. The process used by Robins II to make the chloroadenosine and MTA was an inefficient and expensive non-continuous process.

Thus, it would be highly beneficial to provide more efficient and economical processes for producing chloroadenosine and MTA in high yield. The processes should also be able to provide for the production of chloroadenosine and MTA in situ. A more economical process for the synthesis of chloroadenosine is also desired because chloroadenosine can be used to synthesize MTA and/or MTA analogs.

### SUMMARY OF THE INVENTION

One aspect of the invention is directed to an in situ process of preparing chloroadenosine by:

(a) reacting adenosine in a non-aqueous solvent with a thionyl chloride and a pyridine to form a reaction solution;

(b) exchanging the solvent with a lower alcohol and adding a base to said reaction solution; and

(c) filtering, washing and drying the resulting chloroadenosine.

Preferably, the non-aqueous solvent is any one or a combination of tetrahydrofuran (“THF”), acetonitrile or pyridine, a combination thereof and more preferably is acetonitrile.

Preferably, the lower alcohol is any one or a combination of C₁-C₄ alcohol, and more preferably is methanol.

Preferably, the base is any one or a combination of a carbonate and/or bicarbonate of an alkali metal(s), and alkaline salt or ammonium hydroxide, and more preferably is ammonium hydroxide.

Preferably, the pH of the reaction solution after the solvent is exchanged and the base added is from about 8.8 to about 9.8, and more preferably is about 9. In addition, the reaction solution is preferably cooled to a temperature of about 0° C. after the solvent is exchanged and the base added. The yield of the resulting chloroadenosine preferably is greater than about 70%, and more preferably is greater than about 90%.

A second aspect of the invention is directed to a two-step reaction process for preparing MTA. In the first step of the reaction process, chloroadenosine is prepared in one step as described above. In the second step of the reaction process, chloroadenosine is converted to MTA.

In one embodiment, the chloroadenosine is converted to MTA by reacting the chloroadenosine with alkali thiomethoxide in dimethylformamide. Preferably, the chloroadenosine is converted to MTA by:

(a) adding dimethylformamide and an alkali thiomethoxide to the chloroadenosine to form a second reaction solution;

(b) adding brine to said second reaction solution;

(c) adjusting the pH of the second reaction solution to a pH of from about 6.8 to about 7.2 to form a slurry, and filtering to form a residue;

(d) triturating the residue with water; and

(e) filtering and drying the residue to yield MTA.

Preferably, the alkali thiomethoxide is potassium thiomethoxide or sodium thiomethoxide, and more preferably is sodium thiomethoxide. Preferably, the pH of the slurry is about 7 prior to filtering.

The yield of MTA preferably is greater than about 80%, and more preferably is greater than about 85% based on the initial starting materials.

The invention is also directed to chloroadenosine and MTA made according to the processes described above.

In part, because the conversion of adenosine to chloroadenosine is believed to involve the in situ conversion of cyclic sulfite intermediate to chloroadenosine, the processes of the invention are more efficient and economical than known processes for making chloroadenosine and MTA and result in higher yields of chloroadenosine and MTA.

Additional aspects, features, embodiments and advantages of the present invention will be apparent from the description that follows, or may be learned from practicing or using the present invention.

### DETAILED DESCRIPTION OF THE INVENTION AND PREFERRED EMBODIMENTS

As used herein, the following terms have the defined meanings, unless indicated otherwise.

As used herein, the terms “comprising” and “including” are used herein in their open, non-limiting sense.

The phrase “lower alcohol” is intended to mean a lower alkyl group, i.e. an alkyl group having 1 to 4 carbon atoms (“C₁-C₄”), wherein at least one of the hydrogen atoms is substituted by a hydroxy (—OH) group. The phrase “lower alkyl” refers to a straight- or branched-chain alkyl group having from 1 to 4 carbon atoms in the chain. Exemplary alkyl groups include methyl (Me, which also may be structurally depicted by /), ethyl (Et), n-propyl, isopropyl, butyl, isobutyl, sec-butyl, tert-butyl (tBu), and the like.

The phrase “non-aqueous solvent” means a solvent that contains substantially no water molecules. A broad and common class of non-aqueous solvents are organic solvents. Exemplary non-aqueous solvents include acetonitrile, pyridine, acetone, diethyl ether and tetrahydrofuran (“THF”).

The term “base” means a compound that reacts with an acid to form a salt or a compound that produces hydroxide ions in aqueous solution. Exemplary bases include any one or a combination of a carbonate and/or bicarbonate of an alkali metal(s), an alkaline salt, and ammonium hydroxide. Preferred bases include, but are not limited to, potassium hydroxide, sodium hydroxide, ammonium hydroxide, potassium carbonate, and sodium bicarbonate.

According to one embodiment of the invention, an in situ process for synthesizing chloroadenosine is provided. Without being limited by theory, it is believed that the synthesis of chloroadenonsine proceeds via an in situ conversion of cyclic sulfite intermediate to chloroadenosine. The process includes reacting a suspension of adenosine in non-aqueous solvent with thionyl chloride (preferably about 3 equivalents) and pyridine (preferably about 2 equivalents). The reaction is preferably carried out at a temperature between about −13° C. and about −3° C., more preferably at about −8° C. The non-aqueous solvent may be any suitable non-aqueous solvent for the reaction, and preferably is any one or a combination of THF, acetonitrile or pyridine, and more preferably is acetonitrile. The non-aqueous solvent preferably is present in an amount of about 4 mL/g. The reaction solution is preferably warmed to ambient temperature, for example, a temperature of about 15° C. to about 25° C., while stirring, preferably for more than about 18 hours, and more preferably for about 18 to 25 hours.

Thereafter, the stirring is stopped, the temperature of the solution preferably is maintained at ambient temperature, and the non-aqueous solvent is exchanged for a lower alcohol. In one embodiment, the non-aqueous solvent is exchanged for the lower alcohol by adding water to the reaction solution, removing the non-aqueous solvent, and adding one or more lower alcohol(s) to the solution. Preferably, the water is added in an amount of about 8 mL/g. The non-aqueous solvent preferably is removed by vacuum distillation at a temperature of from about 30° C. to about 40° C., more preferably at about 35° C. The lower alcohol added to the solution preferably is any one or a combination of C₁-C₄ alcohol(s), and more preferably is methanol. The lower alcohol preferably is added in an amount of about 3 mL/g to about 4 mL/g, more preferably in an amount of about 3.5 mL/g.

Thereafter, any base suitable for the reaction is added to the reaction solution, preferably in an amount of about 2 mL/g to about 2.5 mL/g, more preferably in an amount of about 2.5 mL/g. The base preferably is any one or a combination of a carbonate and/or bicarbonate of an alkali metal(s), alkaline salt(s) or ammonium hydroxide, and more preferably is ammonium hydroxide. After the base is added, the solution temperature preferably is maintained from about 35° C. to about 45° C., more preferably at between about 35° C. to about 40° C. The pH of the resulting solution preferably is from about 8.8 to about 9.8, and more preferably about 9. The resulting solution is stirred, preferably for about 1 to about 2 hours, during which time the solution is cooled to room temperature.

Subsequently, the lower alcohol is removed from the reaction solution, preferably by vacuum distillation, and preferably at a temperature range of about 30° C. to about 40° C., more preferably at about 35° C. The resulting solution preferably is then cooled to a temperature of about −5° C. to about 5° C., more preferably to about 0° C., for approximately 1 hour, and subsequently filtered. The resulting chloroadenosine is washed, preferably with a suitable lower alcohol, such as, for example, cold methanol (preferably 1 mL/g), and dried, preferably at a temperature of about 30° C. to about 45° C., more preferably at about 40° C., preferably for about 15 to about 25 hours, more preferably for about 18 hours. The chloroadenosine yield preferably is greater than about 70%, and more preferably is greater than about 90%.

In another embodiment of the invention, a process for preparing MTA is provided wherein the process is a two-step process and can be performed in one reaction vessel. In the first step, adenosine is converted to chloroadenosine as described above. In the second step, chloroadenosine is converted to MTA.

In one embodiment, conversion of chloroadenosine to MTA begins by reacting a stirred suspension of chloroadenosine in DMF with an alkali thiomethoxide. The DMF preferably is present in an amount of about 5 mL/g. The alkali thiomethoxide preferably is any one of or a combination of sodium thiomethoxide or potassium thiomethoxide, and more preferably is sodium thiomethoxide. The alkali thiomethoxide preferably is present in an amount of about 2 to about 2.5 equivalents, more preferably in an amount of about 2.2 equivalents.

The resulting reaction solution is stirred, preferably for about 18 to about 25 hours, more preferably for about 18 hours, and charged with saturated brine (preferably about 15 mL). The solution is then neutralized to a pH of about 6.8 to about 7.2, preferably to a pH of about 7, to result in the formation of a slurry. The solution can be neutralized by the addition of, for example, concentrated HCl or any other suitable acid. The resulting slurry is then cooled to a temperature of about −5° C. to about 5° C., preferably to about 0° C., stirred for about 1 to about 2 hours, preferably for about 1 hour, and then filtered. The resulting residue is triturated with water, for about 1 hour, filtered, and dried for about 12 to about 22 hours, for about 18 hours, at a temperature range of from about 35° C. to about 45° C., preferably at about 40° C., to yield MTA. The yield of MTA preferably is greater than about 80%, and more preferably is greater than about 85% based on initial starting materials.

The abbreviations employed throughout the application have the following meanings unless otherwise indicated:

DMF: dimethylformamide; MTA: methylthioadenosine; SAM: S-adenosylmethionine; THF: tetrahydrofuran; vol: volume.

### EXAMPLES

Materials and Method:

In the method described below, unless otherwise indicated, all temperatures are in degrees Celsius (° C.) and all parts and percentages are by weight, unless indicated otherwise.

Various starting materials and other reagents were purchased from commercial suppliers, such as Sigma-Aldrich Company.

Proton magnetic resonance (¹H NMR) spectra were determined using either a Bruker DPX 300 or a General Electric QE-300 spectrometer operating at a field strength of 300 megahertz (MHz). Chemical shifts are reported in parts per million (ppm) downfield from an internal tetramethylsilane standard. Alternatively, ¹H NMR spectra were referenced to residual protic solvent signals as follows: CHCl₃=7.26 ppm; DMSO-d6=2.49 ppm. Peak multiplicities are designated as follows: s=singlet; d=doublet; dd=doublet of doublets; ddd=doublet of doublet of doublets; t=triplet; tt=triplet of triplets; q=quartet; br=broad resonance; and m=multiplet. Coupling constants are given in Hertz (Hz). Infrared absorption (IR) spectra were obtained using a Perkin-Elmer 1600 series FTIR spectrometer. Elemental microanalyses were performed (by Atlantic Microlab Inc., Norcross, Ga.) and gave results for the elements stated within ±0.4% of the theoretical values. Flash column chromatography was performed using Silica Gel 60 (Merck Art 9385). Analytical thin layer chromatography (TLC) was performed using precoated sheets of Silica 60 F₂₅₄ (Merck Art 5719). Melting points (mp) were determined on a Mel-Temp apparatus and are uncorrected. All reactions were performed in septum-sealed flasks under a slight positive pressure of argon, unless otherwise noted. All commercial reagents were used as received from their respective suppliers.

### Example 1

The following Scheme 1 sets forth a preferred method for preparing chloroadenosine (Compound 2).

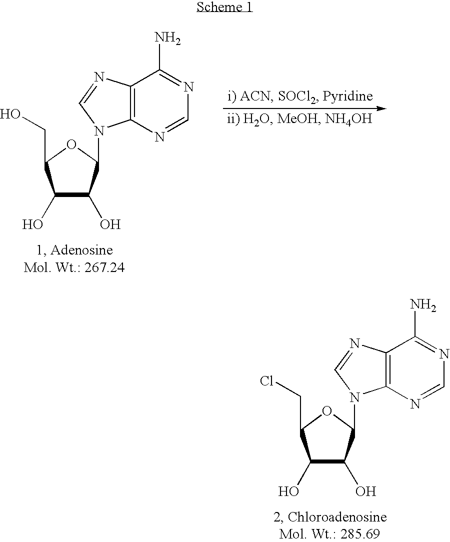

Synthesis of Chloroadenosine:

A 2-liter, 3-neck flask equipped with a mechanical stirrer and a temperature probe was charged with 400 mL of acetonitrile followed by adenosine (100 g, 0.374 mole). The resulting slurry was stirred while cooling to −8° C. with ice/acetone. The reaction was then charged with thionyl chloride (82 mL, 1.124 mole) for over 5 minutes. The reaction was then charged with pyridine (69.8 mL, 0.749 mole), dropwise, for over 40 minutes. The ice bath was removed and the temperature was allowed to rise to room temperature while stirring for 18 hours. The product began to precipitate out of solution. After a total of 18 hours, the reaction was charged with water (600 mL), dropwise. Acetonitrile was removed by vacuum distillation at 35° C. The reaction was then charged with methanol (350 mL). The reaction was stirred vigorously and charged, dropwise, with concentrated NH₄OH (ammonium hydroxide) (225 mL). The addition was controlled to maintain the temperature below 40° C. The pH of the resulting solution was 9. The resulting solution was stirred for 1.5 hours, allowing it to cool to room temperature. After 1.5 hours, 200 mL of methanol was removed by vacuum distillation at 35° C. The resulting clear yellow solution was cooled to 0° C. for 1 hour and then filtered. The resulting colorless solid was washed with cold methanol (100 mL), and then dried at 40° C. under vacuum for 18 hours. The reaction afforded chloroadenosine as a colorless crystalline solid (98.9 g, 92.7%). The NMR¹H indicated that a very clean desired product with a small water peak was produced. ¹H NMR (DMSO-d6): 8.35 (1H), 8.17 (1H), 7.32 (2H), 5.94 (d, J=5.7 Hz, 1H), 5.61 (d, J=6 Hz, 1H), 5.47 (d, J=5.1 Hz, 1H), 4.76 (dd, J=5.7 & 5.4 Hz, 1H), 4.23 (dd, J=5.1 Hz & 3.9 Hz, 1H), 4.10 (m, 1H), 3.35-3.98 (m, 2H).

### Example 2

The following Scheme 2 sets forth a preferred method for preparing MTA (Compound 3).

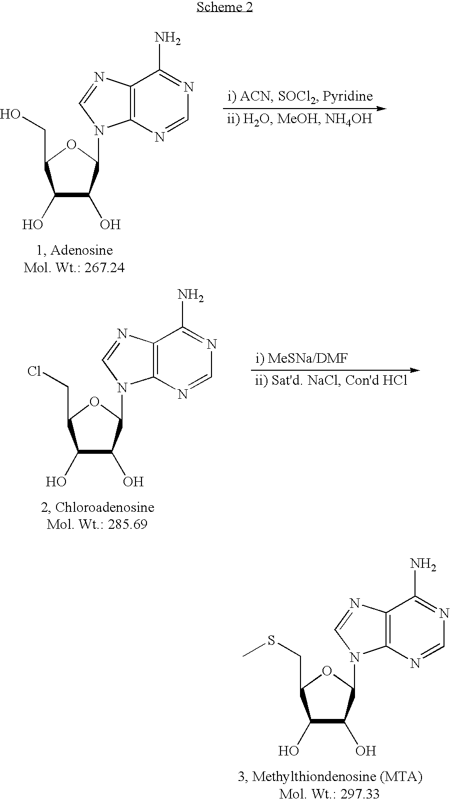

Synthesis of Methylthioadenosine Using Chloroadenosine from Example 1:

A 3-liter, 3-neck flask equipped with a mechanical stirrer and a temperature probe was charged with DMF (486 ml) followed by chloroadenosine (97.16 g, 0.341 mole). The resulting slurry was charged with NaSCH₃ (52.54 g, 0.75 mole), and then stirred with a mechanical stirrer for 18 hours. The slurry was charged with saturated brine (1500 mL) and the pH was adjusted to 7 with concentrated HCl (40 mL). The pH was monitored during addition with a pH probe. The resulting slurry was cooled to 0° C., stirred for one hour with a mechanical stirrer, and filtered. The colorless residue was triturated with water (500 mL) for 1 hour, filtered, and dried under vacuum for 18 hours at 40° C. A colorless solid identified as methylthioadenosine was produced (94.44 g, 93.3% yield from chloroadenosine, 86.5% yield from initial starting materials). The resulting MTA was 99% pure. ¹H NMR (DMSO-d6): 8.36 (1H), 8.16 (1H), 7.30 (2H), 5.90 (d, J=6.0 Hz, 1H), 5.51 (d, J=6 Hz, 1H), 5.33 (d, J=5.1 Hz, 1H), 4.76 (dd, J=6.0 & 5.4 Hz, 1H), 4.15 (dd, J=4.8 Hz & 3.9 Hz, 1H), 4.04 (m, 1H), 2.75-2.91 (m, 2H), and 2.52 (s, 3H).

The practice of the present invention generally employs conventional techniques that are well within the purview of the skilled artisan. Such techniques are explained fully in the literature.

All articles, books, patents, patent applications and patent publications cited herein are incorporated by reference in their entirety. While the invention has been described in conjunction with the foregoing examples and preferred embodiments, it is to be understood that the foregoing description is exemplary and explanatory in nature and is intended to illustrate the invention and its preferred embodiments. Through routine experimentation, one of ordinary skill in the art will recognize apparent modifications and variations that may be made without departing from the spirit of the invention. Thus, the invention is intended to be defined not by the above description, but by the following claims and their equivalents.

What is claimed is:

1. An in situ process for preparing chloroadenosine, consisting essentially of:
(a) reacting adenosine in a non-aqueous solvent with a thionyl chloride and a pyridine to form a reaction solution;
(b) exchanging the non-aqueous solvent with a lower alcohol and adding a base to said reaction solution; and
(c) filtering, washing and drying the resulting chloroadenosine.

2. The process according to claim 1, wherein the non-aqueous solvent is tetrahydrofuran, acetonitrile, pyridine, or a combination thereof.

3. The process according to claim 2, wherein the non-aqueous solvent is acetonitrile

4. The process according to claim 1, wherein the lower alcohol is methanol.

5. The process according to claim 1, wherein the base is a carbonate or a bicarbonate of an alkali metal, an alkaline salt or ammonium hydroxide.

6. The process according to claim 5, wherein the base is ammonium hydroxide.

7. The process according to claim 1, wherein the pH of the reaction solution after step (b) is adjusted to from about 8.8 to about 9.8.

8. The process according to claim 7, wherein the pH is about 9.

9. The process according to claim 1, wherein the reaction solution after step (b) is cooled to a temperature of about 0° C.

10. The process according to claim 1, wherein the yield of chloroadenosine is greater than about 70%.

11. The process according to claim 1, wherein the yield of chloroadenosine is greater than about 90%.

12. A process for preparing methylthioadenosine consisting of:
(1) preparing chloroadenosine by a one-step process consisting essentially of:
(a) reacting adenosine in a non-aqueous solvent with a thionyl chloride and a pyridine to form a reaction solution;
(b) exchanging the solvent with a lower alcohol and adding a base to said reaction solution;
(c) filtering, washing and drying the resulting chloroadenosine; and
(2) converting the chloroadenosine to methylthioadenosine.

13. The process according to claim 12, wherein the non-aqueous solvent is tetrahydrofuran, acetonitrile, pyridine, or a combination thereof.

14. The process according to claim 13, wherein the non-aqueous solvent is acetonitrile.

15. The process according to claim 12, wherein the lower alcohol is methanol.

16. The process according to claim 12, wherein the base is a carbonate or bicarbonate of an alkali metal, an alkaline salt or ammonium hydroxide.

17. The process according to claim 16, wherein the base is ammonium hydroxide.

18. The process according to claim 12, wherein the chloroadenosine is converted to methylthioadenosine by a process comprising reacting the chloroadenosine with an alkali thiomethoxide in dimethylformamide.

19. The process according to claim 18, wherein the alkali thiomethoxide is sodium thiomethoxide or potassium thiomethoxide.

20. The process according to claim 19, wherein the alkali thiomethoxide is sodium thiomethoxide

21. The process according to claim 18, wherein the chloroadenosine is converted to methylthioadenosine by a process comprising:
(a) adding dimethylformamide and an alkali thiomethoxide to the chloroadenosine to form a second reaction solution;
(b) adding brine to said second reaction solution;
(c) adjusting the pH of said second reaction solution to a pH of from about 6.8 to about 7.2 to form a slurry, and filtering said slurry to form a residue;
(d) triturating said residue with water; and
(e) filtering and drying said residue to yield methylthioadenosine.

22. The process according to claim 21, wherein the alkali thiomethoxide is sodium thiomethoxide or potassium thiomethoxide.

23. The process according to claim 22, wherein the alkali thiomethoxide is sodium thiomethoxide.

24. The process according to claim 21, wherein the pH of the slurry is about 7 prior to filtering said slurry.

25. The process according to claim 21, wherein the slurry is cooled to about 0° C. prior to filtering said slurry.

26. The process according to any one of claims 12 or 21, wherein the yield of methylthioadenosine is greater than about 80%.

27. The compound 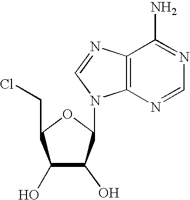
made by the process according to claim 1.

28. The compound 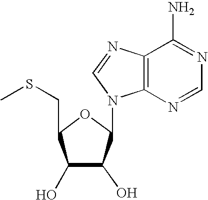
made by the process according to claim 12.
